(19) United States
(12) Patent Application Publication
(10) Pub. No.: US 2010/0262244 A1
(43) Pub. Date: Oct. 14, 2010

(54) Metal Coated Implant

(75) Inventors: Heather Savage-Erickson, Memphis, TN (US); Brian R. Thoren, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/423,621

(22) Filed: Apr. 14, 2009

(51) Int. Cl. A61F 2/44

(52) U.S. Cl. 623/17.16

(57) ABSTRACT

A metal coated implant and a method for using the implant for expanding an intervertebral disc space is provided. The implant comprises a polymer substrate and a metallic coating layer wherein the metallic coating layer is attached to the polymer substrate. There metal coated implant further comprising a biocompatible radiolucent polyetheretherketone (PEEK) material, and a metallic coating layer that is a titanium (Ti) or gold (Au) material. The metal coated implant's radiolucent and radiopaque properties enhance a surgeon's ability to distinctly visualize the metal coated implant to thereby better control and maneuver the delivery, insertion, trajectory, position and orientation of the metal coated implant through the patient anatomical environment and into the vertebral disc space.

### FIELD OF THE INVENTION

The present invention relates to medical devices such as spinal intervertebral implants implanted between adjacent vertebral bodies of a spinal column section, and methods of use, and more particularly to a metal coated implant for intervertebral stabilization comprising radiolucent and radiopaque properties to thereby facilitate visualization of the implant's orientation and/or position during or subsequent to vertebral disc space insertion in a spinal surgical procedure.

### BACKGROUND

The spine is divided into four regions comprising the cervical, thoracic, lumbar, and sacrococcygeal regions. The cervical region includes the top seven vertebral bodies or members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical, thoracic and lumbosacral curve. Within the spine, intervertebral discs are positioned between the vertebral members and permit flexion, extension, lateral bending, and rotation. An intervertebral disc functions to stabilize and distribute forces between vertebral bodies. The intervertebral disc is comprised of the nucleus pulposus surrounded and confined by the annulus fibrosis.

Intervertebral discs and vertebral members are prone to injury and degeneration. Damage to the intervertebral discs and/or vertebral members can result from various physical or medical conditions or events, including trauma, degenerative conditions or diseases, tumors, infections, disc diseases, disc herniations, aging, scoliosis, other spinal curvature abnormalities or vertebra fractures. Damage to intervertebral discs can lead to pain, neurological deficit, and/or loss of motion. Damaged intervertebral discs may adversely impact the normal curvature of the spine, and/or lead to improper alignment and positioning of vertebrae which are adjacent to the damaged discs. Additionally, damaged discs may lead to loss of normal or proper vertebral spacing.

Various known surgical procedures, treatments and techniques have been developed to address medical problems associated with damaged or diseased intervertebral discs. One treatment is a fusion procedure which partially removes the center or nuclear area of a damaged disc and fuses adjacent vertebral members to prevent relative motion between the adjacent vertebral bodies. A section of the disc, annulus and nucleus, is removed or cut out to allow insertion of an implant. The spacer may be used in conjunction with bone graft or allograft material which enables the adjacent vertebrae to grow and fuse together. The spacer assists in maintaining the adjacent vertebrae at a desired spacing height between the vertebrae during the fusion process. The spacer may also assist in imparting desired alignment or lordosis of the adjacent vertebral bodies.

As is well known to persons of skill in the art, there are a variety of structures and configurations which can be used to obtain the desired vertebral body spacing and alignment such as spacers, implants or cages. These structures come in a variety of configurations, features, contours, geometries and sizes depending on the specific medical application or use. Further, as is well known to those of skill in the art regarding established surgical procedures, implants can be inserted from a variety of insertion approaches, including for example anterior, posterior, anterolateral, lateral, direct lateral and translateral approaches.

Known surgical techniques and approaches can be complex, difficult and time consuming depending on implant configuration, and delivery instruments and surgical approaches used. Also, the surgeon must be careful not to injure the spinal cord and neighboring nervous system. During the implant procedure access to the affected disc area may be limited by the person's anatomy, the delivery approach, or the implant's configuration and physical properties. Additionally, it may be difficult for a surgeon to visualize the implant's position and orientation during and after the surgical procedure.

One known approach for implant visualization or fusion assessment is the use of fluoroscopy to visualize an implant with embedded radiopaque tantalum markers during or after a spinal surgical procedure. The implant is typically non-metallic while the tantalum markers are a radiodense metal that is substantially opaque to radiation, and thus more visible relative to the host implant. The tantalum markers typically small rod shaped devices with dimensions that permit imbedding in the host implant. The markers are typically imbedded at implant locations that can convey spatial implant information when viewed with a medical imaging device. The use of fluoroscopy and implants with tantalum markers provides limited visualization of the implant's location and orientation. Subsequent to a surgical implant procedure, computed tomography (CT) scanning or other medical imaging techniques may be used for post-operative imaging, examination and diagnosis of implant position and orientation or vertebral fusion assessment.

A drawback of implants with tantalum markers in post-operative imaging is that visualization remains difficult and limited in part due to the small markers size and the small radiopaque marker image in an X-ray imaging device. Also, the markers do not enable a surgeon to visualize the interface between the vertebrae end plates and the implant, which prevents a surgeon from detecting potential end plate damage or violation of the endplate or endplates due to insertion of the implant into the disc space during the implant procedure. An additional drawback is that the tantalum markers often cause X-ray CT scatter which degrades the images and makes post-operative fusion assessment more difficult.

There is thus a need for an improved implant and method for restoring an intervertebral disc space which addresses the problem of limited visualization of the implant during implant insertion during a spinal surgical procedure, and reduces or eliminates post-operative X-ray CT scatter. There is also a need for an improved intervertebral implant, and method for inserting the implant, between adjacent vertebral bodies using minimally invasive surgical techniques that overcome drawbacks and difficulties in visualizing the orientation and position of the implant during and after implant insertion and in relation to the vertebral body end plates.

### SUMMARY

There is provided a spinal implant for insertion into an intervertebral disc space, the implant comprising a polymer substrate and a metallic coating layer wherein the metallic coating layer is attached to the polymer substrate. There is also provided a metal coated implant where the polymer substrate is a biocompatible radiolucent polyetheretherketone (PEEK) material, and the metallic coating layer is either titanium (Ti) or gold (Au).

There is provided a spinal implant for insertion into an intervertebral disc space, the implant comprising a radiolucent polymer substrate and a radiopaque metallic coating layer attached to the polymer substrate, wherein a spinal implant image is visible via a medical imaging device to thereby determine position or orientation of the spinal implant relative to the vertebral disc space. In a preferred aspect, the polymer substrate is polyetheretherketone (PEEK) and the metallic coating layer is either titanium (Ti) or gold (Au).

There is provided a metal coated implant for insertion into an intervertebral disc space, the metal coated implant comprising a polyetheretherketone (PEEK) polymer substrate having a first surface and a second surface discontinuous from the first surface. The metal coated implant also comprises a biocompatible radiopaque metallic coating layer attached to the polymer substrate covering the first surface and the second surface such that a metal coated implant image is visible via a medical imaging device to thereby enable improved positioning and orienting of the metal coated implant in the vertebral disc space.

There is also provided a method for expanding an intervertebral disc space by inserting a metal coated implant between adjacent vertebrae comprising the steps of removing a portion of an intervertebral disc to create a disc insertion area between the adjacent vertebrae. Selecting a biocompatible metal coated spinal implant comprising a polymer substrate comprising a first surface and a second surface discontinuous from the first surface, and a biocompatible metallic coating layer attached to the polymer substrate and covering the first surface and the second surface. And, inserting the spinal implant into the disc insertion area in the intervertebral disc space to thereby expand the intervertebral disc space.

Disclosed aspects or embodiments are discussed and depicted in the attached drawings and the description provided below.

### BRIEF DESCRIPTION OF THE DRAWINGS

FIG. 1 illustrates a sagittal plane view of a section of a vertebral column and a metal coated spinal implant according to one embodiment of the present disclosure;

FIG. 2A illustrates a view of an interface between an implant substrate and a metal coating layer according to one embodiment of the present disclosure;

FIG. 2B illustrates a view of an implant substrate coated with a first metal coating layer and a second metal coating layer according to another embodiment of the present disclosure;

FIG. 2C illustrates a view of an implant substrate coated with a first metal coating layer and a second metal coating layer according to another embodiment of the present disclosure; and

FIG. 3 illustrates an isometric view of a metal coated spinal implant according to another embodiment of the present disclosure.

### DETAILED DESCRIPTION

The present invention relates to medical devices such as spinal intervertebral implants implanted between adjacent vertebral bodies of a spinal column section, and methods of use, and more particularly to metal coated implants for intervertebral stabilization comprising radiolucent and radiopaque properties to thereby facilitate visualization of the implant's orientation and/or position during or subsequent to vertebral disc space insertion in a spinal surgical procedure. For purposes of promoting an understanding of the principles of the invention, reference will now be made to one or more embodiments or aspects, examples, drawing illustrations, and specific language will be used to describe the same. It will nevertheless be understood that the various described embodiments or aspects are only exemplary in nature and no limitation of the scope of the invention is thereby intended. Any alterations and further modifications in the described embodiments or aspects, and any further applications of the principles of the invention as described herein are contemplated as would normally occur to one skilled in the art to which the invention relates.

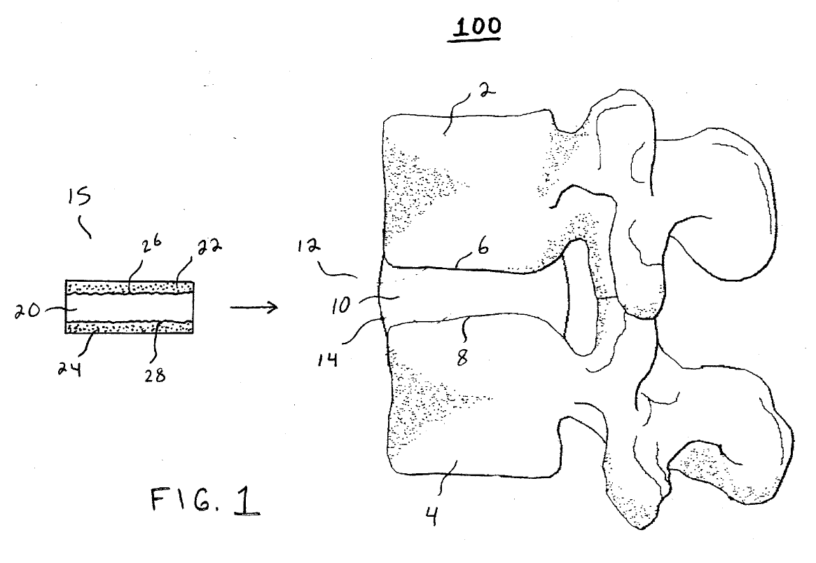

FIG. 1 illustrates a vertebral joint section or motion segment 100 of a vertebral column. The joint section 100 includes adjacent vertebral bodies 2 and 4. The vertebral bodies 2 and 4 include end plates 6 and 8, respectively. An intervertebral disc space 12 is located between the endplates 6 and 8. An intervertebral disc 10 is located in the intervertebral disc space 12 between the adjacent endplates 6 and 8. The intervertebral disc 10 is comprised of an annulus fibrosus or annulus 14 around the disc 10 periphery which substantially surrounds the intervertebral disc space 12, and a nucleus pulposus.

FIG. 1 further depicts a metal coated spinal implant, spacer, device or apparatus 15 which may be inserted into the intervertebral disc space 12. The spinal implant can be used to promote fusion or preserve motion between the vertebral bodies 2 and 4, depending on the specific shape or configuration of the implant used in a surgical procedure. FIG. 1 depicts an implantation technique where the metal coated implant 15 is being delivered to the intervertebral disc space 12 via an anterior approach. Such a spinal implant approach is a known surgical implant procedure and delivery approach. Those of skill in the art will recognize that the spinal implant 15 could also be delivered and inserted through other known surgical approaches, including, a posterior, direct lateral, translateral, posterolateral, or anterolateral or any suitable oblique direction. Some known technique and approaches, among others, include, anterior lumbar interbody fusion (ALIF), posterior lumbar interbody fusion (PLIF), direct lateral lumbar interbody fusion (DLIF) and transforaminal lumbar interbody fusion (TLIF). Further, those of skill in the art will recognize that a spinal implant may be delivered and inserted through known surgical technique and procedures, including: open, mini-open, minimal access spinal technologies (MAST) or other minimally invasive surgical (MIS) techniques. Also, the present metal coated implant 15 is contemplated to be used with typical and existing instruments that are presently used with known surgical techniques.

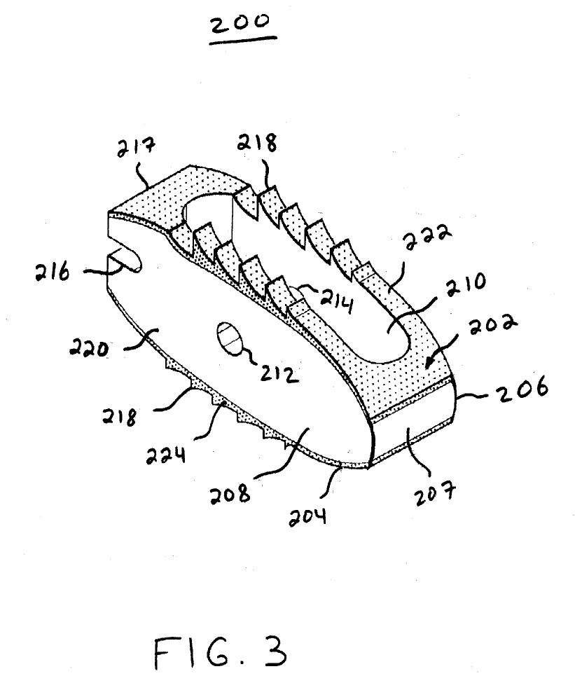

FIG. 1 illustrates a metal coated spinal implant 15 according to one aspect the present disclosure. The metal coated spinal implant 15 comprises an implant substrate 20 and at least one metallic coating layer 22 and 24 attached, deposited or bonded to the implant substrate 20. In the embodiment as shown in FIG. 1, upper and lower metallic coating layers 22 and 24 are attached to the implant's 15 top or cephalad wall or surface 26, and bottom or caudal wall or surface 28. In this embodiment, the upper and lower metallic coating layer 22 and 24 are preferably attached or deposited so as to be two separate metallic coating layers that are discontinuous or non-continuous from one another on the implant's 15 upper and lower wall surfaces 26 and 28. Those of skill in the art will recognize that if desired or needed by a surgeon or a particular medical application, the metal coating layers can also be deposited in a continuous layer. And further, that a single coating layer may also have discontinuities or voids within a layer, for example as shown FIG. 3, discussed below.

The metal coated implant 15 of the present disclosure possesses advantageous radiolucent and radiopaque properties and characteristics which enable or lead to distinct and improved implant visualization when viewed using medical imaging technologies or procedures which use X-rays or other electromagnetic radiation. The metal coated implant's 15 improved visualization enhances a surgeon's ability to distinctly visualize and better maneuver the delivery, insertion, trajectory, position and orientation of the metal coated implant 15 into the vertebral disc space 12 and through the surrounding patient anatomical environment. Also, the distinct visualization enhances a surgeon's ability to determine the implant's location, orientation and position during or subsequent to the implant's 15 insertion into a vertebral disc space 12. The radiolucent and radiopaque properties of the metal coated implant 15 also provide improved visualization which permits a physician to visualize the interface of the implant 15 and vertebral body end plates 6 and 8. The ability to distinctly visualize the interface between the implant 15 and vertebral body end plates 6 and 8 permits a physician to detect (e.g., via fluoroscopy) and minimize or prevent end plate damage or violation of the endplate or end plates caused by or resulting from insertion and deliver of the implant into the disc space 12 during the implant procedure.

Subsequent to implantation of the metal coated implant 15, the radiolucent and radiopaque properties of the metal coated implant 15 also result in the minimization or prevention of X-ray CT scatter when using medical imaging technologies which use X-rays or other electromagnetic radiation. Having images substantially free of X-ray scatter improves visualization which permits improved post-operative assessment capability by a physician, including fusion assessment at the vertebral joint section 100 where the metal coated implant was inserted.

The implant substrate 20 is preferably a solid material with physical properties and characteristics such that the substrate provides sufficient rigidity and structural integrity to substantially maintain desired expansion or separation of the intervertebral disc space 12 when inserted between the vertebral bodies 2 and 4. The implant substrate 20 may be molded, machined, or otherwise manufactured into any shape, geometry, configuration or size that would be needed by a surgeon for a spinal implant procedure. Those of skill in the art will recognize that the implant substrate 20 may take on any shaped desired or required for a particular medical use or application. The implant substrate 20 is preferably sufficiently solid for spinal use and to be able to withstand internal or external forces typically encountered or applied to the spinal joint 100. In one aspect, the implant body or implant substrate 20 is preferably formed of a biocompatible polymer material which has a material stiffness or Modulus of Elasticty (MOE) between cortical bone and cancelleous bone, or similar to cortical bone and cancelleous bone. The biocompatible polymer material is preferably of a kind that will not be reabsorbed during the fusion process. In a preferred aspect, the implant substrate 20 is made of a uniform or homogeneous biocompatible polymer material. However, the implant substrate 20 may, in certain applications, be a mixture or composite of biocompatible materials.

In a preferred aspect, the implant substrate is a polyetheretherketone (PEEK) polymer material. A PEEK material is a radiolucent material which permits transmission of electromagnetic radiation, such as X-rays, emitted by a medical imaging device through the PEEK substrate 20 thereby making the PEEK material radiolucent. Those of skill in the art will recognize that there are other biocompatible radiolucent polymer type materials that can also serve as an implant substrate 20, including, among others, homopolymers, co-polymers and oligomers of polyhydroxy acids, polyesters, polyorthoesters, polyanhydrides, polydioxanone, polydioxanediones, polyesteramides, polyaminoacids, polyamides, polycarbonates, polylactide, polyglycolide, tyrosine-derived polycarbonate, polyanhydride, polyorthoester, polyphosphazene, polyethylene, polyester, polyvinyl alcohol, polyacrylonitrile, polyamide, polytetrafluorethylene, poly-paraphenylene terephthalamide, polyetherketoneketone (PEKK); polyaryletherketones (PAEK), cellulose, carbon fiber reinforced composite, and mixtures thereof.

The metallic coating layers 22 and 24 are preferably comprised of a biocompatible and radiopaque material attached, formed, deposited, sprayed or bonded to the implant polymer substrate 20. In the embodiment shown in FIG. 1, the upper and lower metallic coating layers 22 and 24 are attached to the implant's 15 upper and lower walls 26 and 28, respectively. In one aspect, coating of the implant substrate 20 with a metallic coating layer 22 and 24 can be accomplished by known metallic coating processes, e.g., an Ion Bean Assisted Deposition (IBAD) process or a plasma coating type process. Other known coating processes and techniques may be used depending on the implant substrate 20 and the specific metal coating used including, among others, cathodic arc deposition, sputter deposition, ion beam induced deposition, atmospheric plasma spray, arc spray, cold spray, plasma spray process, high velocity oxy-fuel (HVOF), vacuum plasma spraying (VPS), ion beam sputtering and pulsed laser deposition.

In a preferred aspect, the implant coating layer 22 and 24 is comprised of a biocompatible metal such as titanium (Ti) or Gold (Au), or a combination or mixture of these two metals. The radiodensity of these metals absorb or block electromagnetic radiation, such as X-rays, from passing through the metallic coating comprised of one or both of these metals. Other biocompatible and radiopaque metals are contemplated for the attachment of a metallic implant coating layer 22 and 24 onto an implant substrate 20 including, but are not limited to, Stainless Steel, Cobalt Chrome, Tantalum, Platinum, Tungsten, Silver, Palladium, as well as any mixture, composite, combination an/or alloy of the aforementioned biocompatible metallic materials. The metallic implant coating layers 22 and 24 can be attached, deposited or bonded to a spinal implant substrate 20 of any physical size, shape or configuration.

The radiopaque aspect of the metal coating layer area 22 and 24 on the implant substrate 20 makes the metallic coating layer distinctly visible in a medical imaging device, e.g., on a fluoroscope or in an x-ray static image. The radiopaque metallic layer 22 and 24 in combination with the radiolucent implant substrate 20 result in a distinct or improved implant image which permits a surgeon to have improved implant maneuvering or control for delivery, insertion, trajectory, positioning and orienting during implant insertion into the vertebral disc space and through the patient's surrounding anatomy. Additionally, the radiodensity characteristics and properties of the metal coated layers 22 and 24 on the implant result in the substantial minimization or prevention of X-ray CT scatter in post-operative medical imaging.

The metallic implant coating 22 or 24 can be attached or bonded on a selected portion, section or surface, interior or exterior, of the implant substrate 20, preferably a PEEK substrate. The metallic implant coating layer 22 and 24 can also be attached or bonded over the entirety of surfaces, interior or exterior, of an implant substrate 20. The final metallic coating layer make-up or configuration on an implant substrate 20 will depend on the needs and requirements of a physician or a particular surgical spinal application. The metallic implant coating layers 22 and 24 can also be deposited or formed on the implant substrate 20 in a selected or needed metallic coating pattern determined by a medical application or a physician needs. As such, an implant substrate 20 may be metallically coated along its side surfaces, top or bottom surfaces, or over the entire implant substrate as needed or required by a physician or medical application. In some instances, the decision where to coat the implant substrate 20 may depend on the cost or availability of the selected coating metal. In the case of high expense and limited availability of a coating metal, the implant's substrate can be coated only as is sufficiently necessary for improved visualization.

Where a metallic coating layer is to be formed on a portion, section or surface of the implant substrate 20 or in a specific coating pattern, use of a pattern or design mask permits coating control on the implant substrate 20. A pattern or design mask provides the ability to cover selected surfaces, sections or portions of the implant substrate 20 during manufacturing of the metal coated implant 15. Where the mask covers the underlying substrate 20, no metallic coating will be deposited or formed on the substrate 20. Where the mask does not cover the underlying substrate, a metallic coating will be deposited or formed on the substrate in accordance with the mask patter or design. In this manner, a pattern mask allows for control of where the implant substrate 20 is metallically coated and where it is not. As is known to those of skill in the art, a pattern or design mask can be a sheet or material with a desired pattern or design which can be attached and positioned on a substrate. Methods and means for attachment and bonding of a pattern or design mask on a substrate are well known to those of skill in the art, and use of such known methods and means for attaching and positioning a pattern mask or design plate cut out is contemplated herein.

The use of metallic coating and pattern masks permits the implant substrate 20 to be metallically coated such that the attached or bonded metallic coating follows or complimentarily takes on the contour, shape and configuration of the implant 15. The metallic coating layer 22 and 24 will be able to take on the contour, shape and configuration of the implant 15 whether the implant's surfaces are planar or curved, whether the implant has soft or sharp surface transitions, whether the implant substrate has a simple or complex geometric shape, whether the implant substrate has surface protrusion or tooth configuration patterns or shapes, or whether the implant substrate is for a small or large size implant. Use of a pattern or design mask provides the ability to metallically coat any portion of a substrate's surfaces or the entirety of a substrate's surfaces.

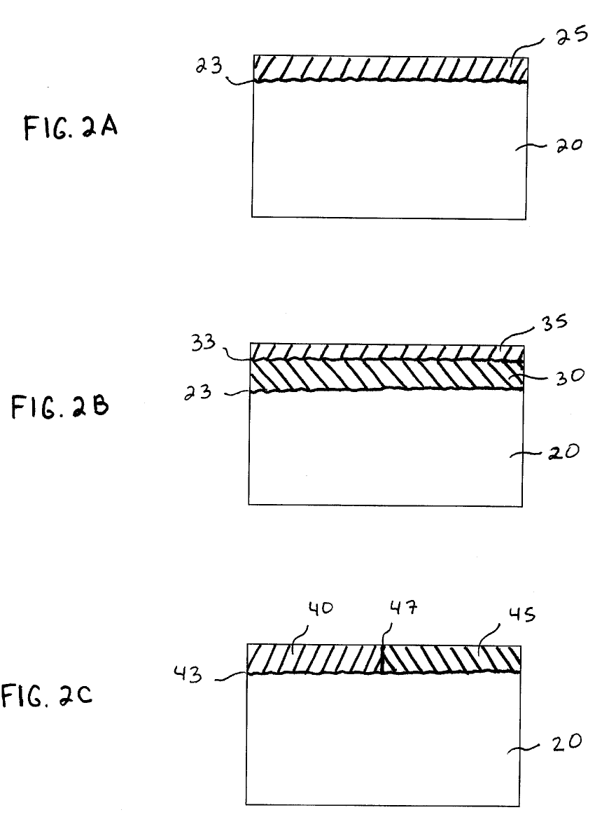

FIGS. 2A-2C illustrate aspects of attaching or bonding one or more metallic coating layers to an implant substrate 20. FIG. 2A illustrates a view of an interface 23 between the implant substrate 20 and a first metal coating layer 25 according to one aspect of the present disclosure. In this aspect, the implant substrate 20 is coated with a single coating layer 25. In the single coating layer option, in a preferred embodiment, the metallic implant coating 25 is selected as titanium (Ti). Titanium (Ti) is the preferred single coating layer material as it possesses good adherence characteristics when attached or bonded directly on a PEEK substrate. Also, titanium (Ti) possesses radiopaque properties such that in combination with the radiolucent PEEK substrate, the physical outline location and orientation of the inserted metal coated PEEK implant 15 can be distinctly visualized and assessed using medical imaging techniques. The radiodensity properties of the titanium (Ti) metal coated PEEK implants enable distinct and improved visualization of the metal coated implant, as discussed above.

In an alternate preferred aspect, gold (Au) may instead be used as the metallic coating layer deposited on the PEEK substrate implant 15. In some instances and applications, gold (Au) may be selected as the material for the coating layer. For example, where certain factors may also or instead be considered or come into play, such as material availability, cost and ease of manufacturing, gold (Au) may instead be selected as the metal coating. Further, as discussed above, other metals may be used to form the single metallic layer instead of the preferred titanium (Ti) or alternate Gold (Au) metallic layer.

FIG. 2B depicts a view of an implant substrate 20 coated with a first metal coating layer 30 and a second metal coating layer 35 according to another aspect of the present disclosure. The two layer or double layer metal coating option also provides distinct and improved implant visualization, as discussed above. As shown in FIG. 2B, a first metal coating layer 30 is deposited on the PEEK substrate, and a second metal coating layer 35 is deposited on the first metal coating layer 30. The two preferred coating metals for coating the implant are titanium (Ti) and gold (Au). The first metal coating layer 30 is preferably selected to be titanium (Ti) and the second metal coating layer 35 is preferably selected to be gold (Au). As discussed above, such a preferred selection of metal layers may be due to titanium's (Ti) good adherence characteristics to the PEEK substrate. Titanium is thus a preferred choice as the first metallic coating layer 30 directly attached adjacent to the PEEK implant substrate. Those of skill in the art will recognize that depending on the material composition of the implant substrate, metal availability, and cost and ease of manufacturing, other metallic coating materials or combination of metal materials may be selected as the first and second metallic coating layers 30 and 35.

FIG. 2C illustrates a view of an implant substrate 20 coated with a first metal coating layer 40 and a second metal coating layer 45 according to another aspect embodiment of the present disclosure. The two layer or double layer metal coating option of FIG. 2C also provides distinct and improved implant visualization, as discussed above. As shown in FIG. 2C, a first metal coating layer 40 and a second metal coating layer 45 are deposited on the PEEK substrate 20. The first metal coating layer 40 and a second metal coating layer 45 are deposited and bonded on the PEEK substrate 20 next to each other instead of layered one on top of another relative to the implant substrate 20 (as was the case in the aspect shown in FIG. 2B). In this embodiment, the two preferred coating metals are titanium (Ti) and gold (Au). The first metal coating layer 40 can be selected to be titanium (Ti) and the second metal coating layer 45 can be selected to be gold (Au), or vice versa. Those of skill in the art will recognize that depending on the material composition of the implant substrate, metal availability, cost and ease of manufacturing, other metallic coating materials or combination of metal materials may be selected as the first and second metallic coating layers 40 and 45. Additionally, a metal coated implant with more than two metal coating layers is also contemplated which can provide distinct and improved implant visualization. In such an embodiment, other biocompatible and radiopaque metals could form one or more of the layers, including Stainless Steel, Cobalt Chrome, Tantalum, Platinum, Tungsten, Silver, Palladium, as well as any mixture, composite, combination an/or alloy of the aforementioned biocompatible metallic materials.

FIGS. 2A-2C also illustrate another aspect of the metallic coating layers formed on an implant substrate 20, the thickness of the metallic coating layers. The metal coating layers can be deposited and bonded onto the implant substrate 20 with selected or desired thickness. The coating thickness of the deposited metallic coating layer can vary depending on the needs or requirements of a surgeon or the medical application where the metal coated implant 15 is to be used. The coating thickness may also depend on how many metallic coating layers are deposited on the implant substrate 20 and the coating metal used.

In one aspect shown in FIG. 2A, where the metal coated implant has one coating layer, the coating thickness may be up to 100 microns or micrometers (μm). In an embodiment, where titanium (Ti) is used as the coating layer, a coating thickness range of between 40-100 microns is preferred. In an embodiment where gold (Au) is used as the single coating layer, a coating thickness range of between 5-10 microns is preferred. These thickness ranges are exemplary coating thickness ranges for two exemplary coating metal materials, titanium (Ti) and gold (Au). Those of skill in the art will recognize that other coating thickness ranges may be used for these coating metal materials, and that other thickness ranges may be used for other coating metals.

Where more than one metallic coating layer is used, for example as shown in FIGS. 2B and 2C, the metal coated implant may use coating layers that have the same or different coating thickness. Further, either the first or second coating layers may have the larger thickness. The determination of whether the layers will have the same or different thickness, or which layer will have the larger thickness will depend on which two coating metals are used, or the needs of the surgeon or medical application. The coating thickness for each coating layer may be up to 100 microns each. In one embodiment, shown in FIG. 2B, the first or middle coating layer 30 is adjacent to the implant substrate 20 and is preferably a titanium (Ti) coating layer with a thickness range of between 40-100 microns. The second coating layer 35 is a gold (Au) coating layer with a coating thickness range of between 5-10 microns. Those of skill in the art will recognize that a metal coated implant 15 can be coated with other metals, and that identical or varying coating thickness ranges may be used instead of the preferred ranges discussed above.

FIG. 3 illustrates an isometric view of a metal coated spinal implant 200 according to one embodiment of the present disclosure. A metal coated implant can be manufactured to any physical shape, configuration or size that may be needed by a surgeon or for a spinal implant procedure or application, FIG. 3 shows one such embodiment. The metal coated implant 200, shown in FIG. 3, comprises an implant substrate 220 with a curved upper wall or top surface 202 and a curved lower wall or bottom surface 204 forming a generally bullet or oval shaped implant 200. The bullet or oval shape configuration of the implant 200 will permit self-distraction of a disc space 12 when or as the implant 200 is inserted into the disc space 12 during a surgical implant procedure. The metal coated implant 200 comprises substantially parallel lateral side walls 206 and 208 which extend generally in an orthogonal direction between the upper and lower walls or top and bottom surfaces 202 and 204. The metal coated implant 200 also comprises a rear wall 217 and a curved front wall or surface 207 which extend between the upper and lower walls or top and bottom surfaces 202 and 204. The metal coated implant 200 also comprises side wall apertures 212 and 214 which extend through the side walls 208 and 210, respectively.

The implant's upper and lower walls 202 and 204, and side walls 206 and 208 define an implant aperture 210 which extends from the upper wall 202 to the lower wall 204. The implant's upper and lower walls 202 and 204 also comprise protrusions, projections or teeth 218 which are integrally formed with and which extend away from the upper and lower wall 202 and 204 surfaces. The teeth 218, as will be discussed below are metallically coated. The implant teeth can provide stability for the implant 200 once it is inserted between the adjacent vertebrae, and assist in preventing the implant 200 from being ejected from the disc space 12. In the aspect shown in FIG. 3, the teeth 218 are generally triangular in shape when viewed from a side profile. Those of skill in the art will recognize that the teeth or protrusions can instead have other shapes, configurations or sizes including, among others, pyramids, triangles, cones, spikes and keels.

The metal coated implant 200 further includes instrument attachment channels 216 which partially extend into the rear wall 217 and the side walls 208 and 210. The instrument attachment channels 216 can be used for insertion and positioning of the metal coated implant 200 in the disc space 12 between the adjacent vertebrae 2 and 4. The instrument attachment channels 216, implant aperture 210, and side wall apertures 212 and 214 could serve to promote bone fusion in-growth once the implant 200 is inserted in place in the disc space 12. Prior to or during implantation of the metal coated implant 200 into the disc space 12, the attachment aspect 216, implant aperture 210, and side wall apertures 212 and 214 may also be filled with a graft material. The graft material may be composed of any type of material that has the ability to promote, enhance and/or accelerate the to promote bone growth and fusion or joining together of the vertebral bodies 2 and 4 by one or more mechanisms such as, osteogenesis, osteoconduction and/or osteoinduction. This may include allograft material, bone graft, bone marrow, a demineralized bone matrix putty or gel and/or any combination thereof. The filler material may promote bone growth through and around the apertures to promote fusion of the intervertebral joint 100. Those of skill in the art will recognize that the use of filler graft material is optional, and it may or may not be used depending on the needs or requirements of a physician or a medical procedure.

The metal coated implant 200, shown in FIG. 3, also comprises two non-continuous metallic coating layers 222 and 224 attached, deposited or bonded to the polymer substrate 20. In particular, in this embodiment, the metal coated layers 222 and 220 are attached or bonded to the upper wall or top surface 202 and the lower wall or bottom surface 204, respectively. FIG. 3 also shows that in this embodiment, the metal coated layers 222 and 220 extend over the lateral side walls 206 and 208, the curved front wall or surface 207, and the rear wall 217 near and along the periphery of the upper wall or top surface 202 and the lower wall or bottom surface 204, respectively. The upper and lower metallic coating layer 222 and 224 are preferably attached or deposited as two separate metallic coating layers on the implant's 200 upper and lower wall surfaces 226 and 228 as discontinuous or non-continuous coating layers. In this embodiment, the single coating layers 222 and 224 are preferably titanium (Ti) with a coating thickness range of between 40-100 microns, although as discussed above other metals and thickness ranges may be used. Those of skill in the art will recognize that if desired or needed by a surgeon or a particular medical application, the metal coating layers can also be deposited in a continuous layer. And further, that a single coating layer may also have discontinuities or apertures 210 within a metal coating layer, as shown FIG. 3.

In alternate aspects and embodiments, the metal coated implant may have upper and lower walls which are angled relative to one another to achieve a desired or selected kyphosis, lordosis, or lateral wedge effect. In other embodiments, an implant wall or surface may extend obliquely from an adjacent wall rather than orthogonally. Also, any of the implant's walls could be tapered, sloped, angled, or curved, including covex, bi-convex and concave curving, depending on a particular medical application need. The metal coated implant may also be made to have any size, shape and geometry for mating with existing insertion or improved instruments.

The metal coated implant 15 and 200 may be implanted in the disc space 12 using known methods, procedures and approaches, including an anterior, posterior, translateral, direct lateral or any suitable oblique direction, as those of skill in the art will recognize. Further, a spinal implant may be delivered and inserted through known surgical technique and procedures, including: open, mini-open, minimal access spinal technologies (MAST) or other minimally invasive surgical (MIS) techniques.

In one approach, the metal coated PEEK implant 15 and 200 is inserted via an anterior approach. In one aspect, the metal coated implant 15 or 200, shown in FIGS. 1 and 3, will have a selected or desired physical shape and size for use in a spinal medical procedure. Those of skill in the art will readily recognize that the metal coated implant 200 may take on any shaped desired or required for a particular medical use or application. Further, those of skill in the art will recognize that the metal coated implant can also be a dynamic vertebral implant device, with varying shape and size depending on the medical application where the implant used.

Prior to insertion, known medical instruments and tools may be used to prepare the intervertebral disc space 12, including specialized pituitary rongeurs and curettes for reaching the nucleus pulposus or other area in the disc space 12. The disc space 12 may be prepared with a partial or complete discectomy. Ring curettes may be used as necessary to scrape abrasions from the vertebral endplates 6 and 8. Using such instruments, a location which will accept the metal coated implant 15 and 200 is prepared in the disc 10 and disc space 12. Those of skill in the art will recognize that the metal coated implant 15 and 200 may be positioned at any desired location between the adjacent vertebral bodies 2 and 4 depending on the surgeon's need and the performed surgical procedure or medical application.

The implant is then inserted into the prepared disc space 12 using insertion instruments which are appropriate with the shape and configuration of the metal coated implant and surgical procedure to be used. A medical imaging technique and device may be used to distinctly visualize the metal coated implant 15 during the insertion procedure by taking advantage of the metal coated implant's radiolucent and radiopaque properties. During the insertion step, the enhanced metal coated implant visualization will permit the surgeon to better maneuver and control the trajectory, position and orientation of the metal coated implant 15 into the vertebral disc space 12 and through the surrounding patient anatomical environment.

The metal coated implant 15 or 200 is then delivered into the intervertebral disc space 12 and positioned in a selected location and orientation between the end plates 6 and 8 of the adjacent vertebral bodies 2 and 4. The metal coated implant is inserted into the disc space 12 such that the upper wall 202 is positioned adjacent to the upper vertebral endplate 6 and the lower wall 204 is positioned adjacent to the lower vertebral endplate 8. The teeth projections 218 may engage the vertebral endplates 6 and 8 to provide stability to the implant 200. Once implanted, the upper metal coating layer 222 attached to the upper substrate wall 202 will contact the upper vertebral end plate 6 to form an interface between the metal coated implant 200 and the upper vertebral body 2. Also, the lower metal coating layer 224 attached to the lower substrate wall 204 will contact the lower vertebral end plate 8 to form an interface between the metal coated implant 200 and the lower vertebral body 4. After the insertion of the metal coated implant 200 between the vertebral bodies 2 and 4 has been completed, the metal coated device 200 may promote the fusion or joining together of the vertebral bodies 2 and 4.

While embodiments of the invention have been illustrated and described in detail in the present disclosure, the disclosure is to be considered as illustrative and not restrictive in character. All changes and modifications that come within the spirit of the invention are desired to be protected and are to be considered within the scope of the disclosure.

1. A spinal implant for insertion into an intervertebral disc space, the implant comprising:
a radiolucent polymer substrate; and
a radiopaque metallic coating layer attached to the polymer substrate;
wherein a spinal implant image is visible via a medical imaging device to thereby enable determination of position or orientation of the spinal implant relative to the vertebral disc space.

2. The implant of claim 1, wherein the polymer substrate is polyetheretherketone (PEEK).

3. The implant of claim 1, wherein the metallic coating layer comprising one or more of titanium (Ti) and gold (Au).

4. The implant of claim 1, wherein the metallic coating layer has a thickness range of between 40 to 100 microns (μm).

5. The implant of claim 1, wherein the metallic coating layer has a thickness range of between 5 to 40 microns (μm).

6. The implant of claim 1, wherein the metallic coating layer covers a first surface of the polymer substrate.

7. The implant of claim 6, wherein the metallic coating layer covers a second surface of the polymer substrate noncontinuous from the first surface.

8. The implant of claim 7, wherein
the first surface is a top substrate surface, and
the second surface is a bottom substrate surface.

9. The implant of claim 1, further comprising at leas one additional metallic coating layer.

10. The implant of claim 9, wherein the at least one additional metallic coating layer is adjacent to the metallic coating layer.

11. The implant of claim 10, wherein
the metallic coating layer is titanium (Ti), and
the at least one additional metallic coating layer is gold (Au).

12. A metal coated implant for insertion into an intervertebral disc space, the metal coated implant comprising:
a polyetheretherketone (PEEK) polymer substrate comprising a first surface and a second surface discontinuous from the first surface; and
a biocompatible radiopaque metallic coating layer attached to the polymer substrate covering the first surface and the second surface;
wherein a metal coated implant image is visible via a medical imaging device to thereby enable improved positioning and orienting of the metal coated implant in the vertebral disc space.

13. The implant of claim 12, wherein the metallic coating layer has a thickness range of between 40 to 100 microns (μm).

14. The implant of claim 12, wherein the metallic coating layer has a thickness range of between 5 to 40 microns (μm).

15. The implant of claim 12, wherein
the first surface is a top substrate surface, and
the second surface is a bottom substrate surface.

16. The implant of claim 12, further comprising at leas one additional metallic coating layer adjacent to the metallic coating layer.

17. The implant of claim 16, wherein
the metallic coating layer is titanium (Ti), and the at least one additional metallic coating layer is gold (Au).
